(19) United States
(12) Patent Application Publication
(10) Pub. No.: US 2026/0071157 A1
(43) Pub. Date: Mar. 12, 2026

(54) APPLE CIDER VINEGAR FORMULATION

(71) Applicant: BRAGG LIVE FOOD PRODUCTS, LLC, Santa Barbara, CA (US)

(72) Inventors: LOUIS IZUCHUKWU NDIFE, Santa Barbara, CA (US); JONES YIU CHUNG CHAN, Santa Barbara, CA (US); JOSE EMILIO VILLARREAL, Santa Barbara, CA (US); DIANE HUMPHREY KULL, Santa Barbara, CA (US)

(21) Appl. No.: 19/388,110

(22) Filed: Nov. 13, 2025

Related U.S. Application Data
(63) Continuation-in-part of application No. 18/542,529, filed on Dec. 15, 2023.
(60) Provisional application No. 63/500,845, filed on May 8, 2023.

(51) Int. Cl. C12J 1/00; A61K 31/19; A61K 31/198; A61K 31/593; A61K 31/718; A61K 33/30; A61K 36/00; A61K 38/16

(52) U.S. Cl. CPC C12J 1/00; A61K 31/19; A61K 31/198; A61K 31/593; A61K 31/718; A61K 33/30; A61K 36/00; A61K 38/16

(57) ABSTRACT

There is disclosed a formulation comprising apple cider vinegar and an acetate, in various ratios and measures, which form a part of a dose for introducing apple cider vinegar to the human body for health benefits.

### RELATED APPLICATION INFORMATION

This application is a continuation in part of U.S. application Ser. No. 18/542,529, filed Dec. 15, 2023 entitled “ACV SUPPLEMENT FORMULATION AND METHOD OF PRODUCING” which claims priority to U.S. Provisional Application No. 63/500,845 filed May 8, 2023 entitled “ACV SUPPLEMENT FORMULATION AND METHOD OF PRODUCING” of which is incorporated herein by reference in its entirety.

### NOTICE OF COPYRIGHTS AND TRADE DRESS

A portion of the disclosure of this patent document contains material which is subject to copyright protection. This patent document may show and/or describe matter which is or may become trade dress of the owner. The copyright and trade dress owner has no objection to the facsimile reproduction by anyone of the patent disclosure as it appears in the Patent and Trademark Office patent files or records, but otherwise reserves all copyright and trade dress rights whatsoever.

### BACKGROUND

### Field

The disclosure is for a supplement formulation containing vinegar and salts of acetic acid in various supplement delivery forms to deliver a meaningful or efficacious amount of acetic acid to a person. The formulation is one which may support health benefits such as but not limited to healthy blood glucose, cholesterol, and weight levels.

### Description of the Related Art

In ancient times, apple cider vinegar (ACV) was utilized for a variety of purposes across different cultures. In ancient Egypt, around 3000 BCE, vinegar was used for both culinary and medicinal purposes. The Babylonians, around 5000 BCE, reportedly used vinegar as a preservative and condiment. In ancient Greece, Hippocrates prescribed a mixture of honey and vinegar for various ailments. The Romans produced vinegar by fermenting wine, and it was a common condiment in their food. In Asia, especially in China, vinegar was used for culinary purposes and as a preservative. ACV's acidity made it useful in food preservation, and it was also believed to have health benefits, ranging from digestion aid to wound disinfection.

In the early 1900s, apple cider vinegar became a popular household staple with a rich history of different uses. ACV's reputation as a natural remedy persisted, with many households incorporating it into traditional health remedies. ACV was often employed for its purported benefits in digestion, acting as a tonic to alleviate indigestion and promote gut health. Furthermore, its acidic properties made it a sought-after ingredient for preserving and pickling fruits and vegetables before the advent of modern refrigeration methods. Beyond culinary applications, apple cider vinegar found a place in beauty routines, as it was believed to enhance the skin and hair's natural shine. This era marked a continuation of ACV's longstanding presence in homes, embodying a multi-faceted role as a culinary, medicinal, and cosmetic resource.

Prevailing science indicates that consuming one or more tablespoons, or such other amount, of vinegar such as apple cider vinegar provides certain health benefits. The active component of vinegar is acetic acid. A clinical dose has been shown to be as low as 500 mg of acetic acid. However, there may be barriers to consuming an efficacious amount of vinegar including taste and enamel corrosion. Furthermore, the concentration of acetic acid in vinegar in either powder or liquid form is insufficient to deliver efficacious dosing in a supplement form without causing compliance issues. There are no supplements, as far as can be determined, in the marketplace that provide efficacious dosing of acetic acid.

Health benefits of Apple Cider Vinegar (ACV) drinks have been reported widely in scientific literature. Some of the benefits include providing support for healthy blood glucose levels, healthy blood lipid profiles, and healthy body weight maintenance (Ostman et al. 2005, Carol S. Johnston et al., 2010a (trial 1), Tomoo Kondo et. al., 2009, Carmelo Panetta et al., 2013, Darzi et al., 2014). It is also known that the active component of ACV that is responsible for most of these health benefits is the acetic acid.

Acetic acid is generally produced in the process of manufacturing ACV by the action of acetic acid bacteria that are inherent in the apple or its unpasteurized juice. ACV dosage is generally recommended to be 1 to 2 table spoonful (TBSP) per day, to be consumed with a meal and plenty of water. Since a TBSP is about 15 mL, with a total acetic acid content of about 5% and a density of about 1.0, a TBSP contains about 15 g of ACV liquid or about 750 mg of acetic acid. Some of the human clinical studies listed above have shown that intake of about 750 mg acetic acid per day would support those health benefits both in healthy and unhealthy individuals. Recent advents in analytical chemistry and double blind clinical studies have allowed modern researchers to not only pinpoint the exact cause of apple cider vinegar's health benefits, but also formulate modern day remedies that allow for users to obtain the health benefits of ACV without the negative taste.

### SUMMARY

Disclosed herein are compositions of vinegar and salts of acetic acid in various supplement delivery forms that deliver a meaningful or efficacious amount of acetic acid to a person which may be used to support health benefits such as but not limited to healthy blood glucose, cholesterol, and weight levels.

Disclosed herein is a composition comprising apple cider vinegar (ACV) in a solid form and a salt of acetic acid, the ratio of apple cider vinegar to the salt of acetic acid being from about 1.5:1 to 0.5:1, and in some embodiments, from about 1.5:1 to 0.0:1.

Disclosed herein is a nutritional supplement composition comprising apple cider vinegar (ACV) in a dry powder form and a salt of acetic acid, the ratio of apple cider vinegar to the salt of acetic acid being from about 1.5:1 to 0.5:1, and in some embodiments, from about 1.5:1 to 0.0:1, the nutritional supplement being deliverable in a dose where the amount of acetic acid is between about 500 mg and about 2000 mg, and in some embodiments, between about 500 mg and about 1500 mg.

Disclosed herein is a composition comprising a dry solid blend in approximately 1 to 1 ratio of sodium di-acetate and dry apple cider vinegar powder, an excipient, and a discretionary ingredient selected for its ability to enhance the health of a user. In some embodiments, the amount of dry apple cider vinegar powder in the blend is about 313 mg-940 mg. In some embodiments, the amount of sodium di-acetate in the blend is about 250 mg-750 mg. In some example embodiments, the amount of dry apple cider vinegar powder in the blend is about 830 mg. In some example embodiments, the amount of sodium di-acetate in the blend is about 847 mg.

The term “excipient” means a pharmacologically inactive component such as a diluent, disintegrant, carrier, etc., of a nutritional supplement composition. Non-limiting examples of an excipient include flow agents and anti-caking agents. Non-limiting examples of discretionary ingredients include vitamins, minerals, herbs, specialty ingredients, amino acids and/or proteins.

Disclosed herein are methods of making the described composition. The method may comprise making apple cider vinegar (ACV) powder. The method may comprise making a salt of acetic acid such as sodium diacetate. The salt of acetic acid may be in the form of particles. The method may comprise adding a salt of acetic acid to the ACV powder.

The method of making apple cider vinegar (ACV) powder may comprise filtering liquid apple cider vinegar (ACV) to remove selected solid particles and impurities to form a purified ACV, diluting the purified ACV to achieve solid content suitable for spray drying, feeding the diluted purified apple cider vinegar solution into a spray dryer chamber, atomizing the solution in the spray dryer to form fine droplets of the solution, spraying the droplets of apple cider vinegar on to a carrier, collecting the dried particles formed, and separating the solid particles from the air in a drying chamber to form a powder. The powder may be sized and optionally packaged.

In some embodiments, the salt of acetic acid comprises sodium diacetate. The sodium diacetate may in some embodiments be produced by reacting acetic acid with metal bicarbonate such as but is not limited to sodium bicarbonate. For example, in some embodiments, the method involves reacting acetic acid (CH₃COOH) with sodium bicarbonate (NaHCO₃) in a controlled environment to form sodium acetate (CH₃COONa), water (H₂O), and carbon dioxide (CO₂); concentrating the sodium acetate solution obtained from the reaction and allowing to cool such that sodium diacetate crystals start to form; separating the crystals from the remaining solution through filtration; and drying the sodium diacetate crystals to remove moisture. The sodium diacetate crystals may be milled and/or packaged to achieve a selected particle size.

### DESCRIPTION OF THE DRAWINGS

FIG. 1 is a drawing of a chemical structure of acetic acid.

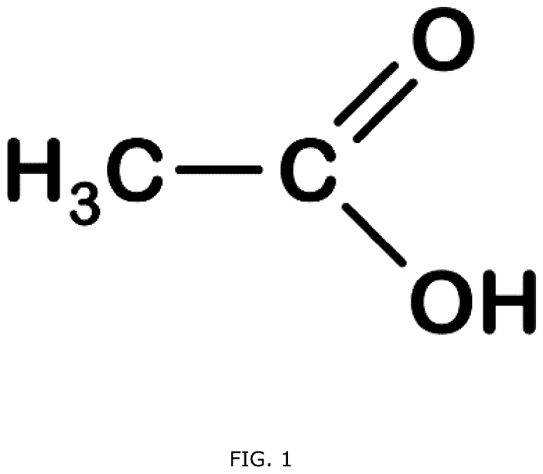

FIG. 2 is a drawing of a chemical synthesis of vinegar from apples.

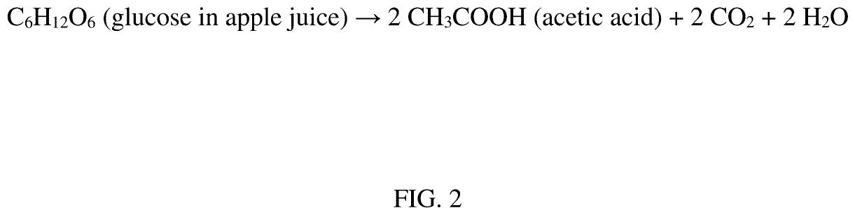

### DETAILED DESCRIPTION

Disclosed herein are multiple embodiments for producing and using Apple Cider Vinegar (ACV), preferably in a solid dose such as a dry powder, a blend of ACV powder and/or an appropriate salt of acetic acid (or acetate) in a capsule form to deliver an efficacious amount of acetic acid of about 500 mg to about 2000 mg acetic acid, and in some embodiments about 500 mg to about 1250 mg and in some embodiments, about 500 mg to about 800 mg per dose of 1 to 8 capsules, and in some embodiments, 1 to 5 capsules and in some embodiments, 1 to 4 capsules. The above amounts and dosages are some example embodiments of the formulation, and formulations with other amounts and dosages would fall within this disclosure.

Sodium diacetate is a chemical compound with the molecular formula of C₄H₇NaO₄. It is a white crystalline powder that is highly soluble in water and has a melting point of 324° C. Sodium diacetate is a combination of an approximately 50:50 mixture of acetic acid and sodium acetate (the sodium salt of acetic acid). When dissolved in water, sodium diacetate disassociates into acetic acid and sodium acetate ions. Acetic acid is a weak organic carboxylic acid that can exist in water as the acid (protonated) form or as a salt (deprotonated) form depending on the pH of the medium. At pH 1-3, it is mainly in the carboxylic acid form. Acetic acid can be comprised of protonated or de-protonated forms depending on pH of the medium. At a neutral pH of 7, it is mainly in the salt form, sodium acetate. Acetic acid is the main nutrient found in vinegar and is credited for many of the health benefits associated with vinegar consumption.

In one embodiment dry ACV powder can be achieved by tower spray-drying ACV liquid slurry, or by vacuum-assisted freeze-drying of ACV in the presence of an appropriate cryoprotectant. Blending ACV powder with salts of acetate, however, may be a better option for operational, cost of manufacture, health and other reasons. In some example embodiments, suitable salts of acetic acid that can be used include sodium acetate, calcium acetate potassium acetate, sodium di-acetate, potassium di-acetate, acetate, iron acetate, magnesium acetate and/or zinc acetate. The ability to remain in a dry state at room temperature, ease of handling by plant operators and the capability to deliver adequate amounts of acetate molecules per gram of ingredient make sodium di-acetate and potassium di-acetate better preferred in some embodiments, with sodium di-acetate being the most preferred acetic acid salt in some embodiments.

Consuming about 750 mg of acetic acid or more from liquid ACV is generally known for its repulsive sour taste. But 750 mg or more dosing, if put in the correct delivery form, can alleviate this sour taste. Mixture formulations of vinegar (liquid or powder) and salts of acetic acid may be selected from one or more of, or a combination of: Vinegar 0.0%, Salts of acetic acid 100.0%; in some embodiments, Vinegar 0.1%, Salts of acetic acid 99.9%; and in some embodiments, Vinegar 50.1%, Salts of acetic acid 49.9%. Other ratios of such mixture formulations may be used and fall within the disclosure.

Potential supplement forms to provide about 750 mg acetic acid or more dosing forms include: solid forms such as but are not limited to tablet form (such as chewable, swallowable, fast melt or melts); gummy form (such as chewable, suckers); and capsule including two-piece capsule, and softgels. The chewables and suckers cannot necessarily solve the sour flavor of acetic acid, especially if one must control the amount of sugar per dose. In the swallowable form, the two-piece capsules and softgels are not only easier to consume, but may be better preferred than tablets by consumers. Additionally, powders or powder sachet as a drink mix may also be used. Furthermore, tablets may have a greater chance of contacting taste buds on the tongue to elicit sour note sensations in some sensitive individuals. Two-piece capsules or softgel delivery forms may therefore afford best protection for the user against the sour taste of the acetic acid. They are easier to swallow and may also be sugar-free. Additionally, acetic acid may be assayed using HPLC as a titratable acid test may not be specific enough as it picks up other organic acids.

Studies have revealed that some consumers are sensitive to the number of capsules they must take to have efficacious levels of the bioactive ingredient in their systems. This is understandable given that some of these consumers are already taking other solid dose medications throughout the day. In various embodiments, the present disclosure therefore requires 1 to 8 capsules per dose, and in some embodiments, 1 to 6 capsules per dose, and in some embodiments, 1 to 4 capsules per dose. The disclosure, however, may cover dosages and capsule numbers and amounts which differ from the above.

The following tables list several examples of a formulation for a serving or regimen of three capsules:

TABLE 1

| | |
|---|---|
| Core Ingredient | About 1677 mg of Bragg proprietary blend |
| | in approximately 1 to 1 ratio of sodium di- |
| | acetate (about 847 mg) and dry ACV |
| | powder (about 830 mg). |
| Discretionary Ingredients | Excipients as required and necessary. |

TABLE 2

| | |
|---|---|
| Core Ingredients | About 1677 mg of Bragg proprietary blend |
| | in approximately 1 to 1 ratio of sodium di- |
| | acetate (about 847 mg) and dry ACV |
| | powder (830 mg). |
| | Excipients as required and necessary. |
| Discretionary Ingredients | Thiamine, Riboflavin, Niacin, Biotin, |
| | Pantothenic acid, Pyridoxine, Vitamin B12, |
| | Folic Acid, Vitamin C, Vitamin D, Vitamin E, |
| | Vitamin K, Calcium, Potassium, Phosphorus, |
| | Sodium, Magnesium, Iron, Zinc, Copper, |
| | Selenium, Molybdenum, Iodine. |

TABLE 3

| | |
|---|---|
| Core Ingredients | About 1677 mg of Bragg proprietary blend |
| | in approximately 1 to 1 ratio of sodium di- |
| | acetate (about 847 mg) and dry ACV |
| | powder (about 830 mg). |
| | Excipients as required and necessary |
| Discretionary Ingredients | Thiamine, Riboflavin, Niacin, |
| | Pantothenic acid, Biotin, Vitamin B12 |

TABLE 4

| | |
|---|---|
| Core Ingredients | About 1677 mg of Bragg proprietary blend |
| | in approximately 1 to 1 ratio of sodium di- |
| | acetate (about 847 mg) and dry ACV |
| | powder (about 830 mg). |
| | Excipients as required and necessary |
| Discretionary Ingredients | Vitamin D3, Zinc. |

TABLE 5

| | |
|---|---|
| Core Ingredients | About 1677 mg of Bragg proprietary blend |
| | in approximately 1 to 1 ratio of sodium di- |
| | acetate (about 847 mg) and dry ACV |
| | powder (about 830 mg). |
| | Excipients as required and necessary |
| Discretionary Ingredients | Thiamine, Riboflavin, Niacin, |
| | Pantothenic acid, Vitamin B12 |

TABLE 6

| | |
|---|---|
| Core Ingredients | About 1677 mg of Bragg proprietary blend |
| | in approximately 1 to 1 ratio of sodium di- |
| | acetate (about 847 mg) and dry ACV |
| | powder (about 830 mg). |
| | Excipients as required and necessary |
| Discretionary Ingredients | Organic Ashwagandha extract. |

While specific amounts and measures have been provided in the above examples, generally for each of a three capsule serving, other amounts may also be used, depending on factors such as the number of capsules or doses which are being formed. Whatever amount is selected, the approximately 1 to 1 ratio of sodium-diacetate and dry ACV powder may be maintained. Further, acetates other than sodium-diacetate, including those mentioned in this disclosure, may be used instead of, or in various combination and selections, in the formulations.

Additionally, for formulations generated from the table above it should be understood that about 1877 mg of proprietary blend (or other suitable amounts) may be used instead of 1677 mg. This is because the acetate and ACV powder may be adjusted accordingly for all the examples. Finally, nutrients such as vitamins, minerals, herbs, functional oils, proteins, amino acids and other specialty supplement actives may be combined with the proprietary blend listed above.

An example embodiment of the invention includes a nutritional supplement comprising a powder formulation of dry ACV and an acetate salt configured to deliver at least 500 mg of acetic acid per dose per day. Another example embodiment of the invention could include the powder formulation of a nutritional supplement comprising a powder formulation of dry ACV and an acetate salt wherein the powder also includes: Organic Apple Cider Vinegar Powder 3-8%, Acetic Acid (313 mg-940 mg); Sodium Diacetate food grade crystal (250 mg-750 mg); Vitamin D3 Cholecalicferol 100,000 iu (0.01 mg-0.03 mg); Zinc Oxide (80% ZN) powder (4.3 mg-13 mg); Silicon Dioxide powder (3.2 mg-9.6 mg); Pea Starch powder (24.4 mg-73.3 mg).

It should be understood that the acetate salts listed above could include acetate salt, sodium, di-sodium, and di-potassium. Calcium salts and/or zinc salts may also be used. Additionally, another version of the invention could include a formulation wherein the nutritional supplement comes in the form of a solid two piece hard shell or softgel capsule.

A method for delivering is also disclosed herein. One potential method could include a method of delivering 500 mg of acetic acid or greater, such as in the range of 500 mg to 2000 mg of acetic acid, to a person in need thereof comprising: ingesting a nutritional supplement comprising a powder formulation of dry ACV and an acetate salt. The method above could further comprise the delivery of the dose in 1 to 8 capsules, and in some embodiments, in 1 to 4 capsules, a day. The method above could further comprise the use of nutrients including the 23 major vitamins and minerals or a subset of them, or any combination of selected nutrients, based on a specific application or use of the formulation. In some embodiments, other accessory supplement active ingredients such as vitamins, minerals, herbs, specialty ingredients, amino acids, proteins and any combinations thereof may be added to the formulation comprising ACV and/or acetate salt.

### Examples of Methodology

The following examples represent a selection of many types of methods for making the composition or components thereof.

### ACV Powder Process

In one embodiment, spray drying is used to turn apple cider vinegar liquid form into a dry powder while maintaining its properties. The following comprises a basic overview of the process:
(1) Preparation of Apple Cider Vinegar (ACV): Ensure that the apple cider vinegar is properly prepared and filtered to remove any solid particles or impurities that could affect the spray drying process.
(2) Dilution (if necessary): Depending on the concentration of the apple cider vinegar, it may need to be diluted with water to achieve the desired solids content for spray drying. This step is important for controlling the viscosity of the solution, which affects the atomization process.
(3) Atomization: The diluted apple cider vinegar solution is fed into a spray dryer chamber. Atomization can be achieved using various methods such as rotary atomization, pressure nozzles, or centrifugal atomizers. The goal is to create fine droplets of the liquid.
(4) Drying: Once atomized, the droplets of apple cider vinegar are sprayed on to a carrier such as, for example, maltodextrin.
(5) Particle Collection: As the dried particles are formed, they are carried out of the drying chamber with the airflow. The solid particles are then separated from the air using cyclones or bag filters.
(6) Packaging: The resulting powder is then sifted, sized and packaged.

Throughout the process, it is important to monitor parameters such as inlet air temperature, feed rate, atomization pressure, and airflow to ensure optimal drying conditions and product quality. Additionally, proper hygiene and sanitation measures should be maintained to prevent contamination of the final product.

### Sodium Diacetate Process

The sodium diacetate is typically produced through the reaction of acetic acid (vinegar) which may include generic vinegar and/or specialty vinegar such as ACV with sodium bicarbonate (baking soda). The following is a representative overview of the process:
(1) Preparation of Acetic Acid (Vinegar) and/or specialty vinegar such as ACV: Acetic acid is the main component of vinegar.
(2) Neutralization Reaction: Acetic acid (CH₃COOH) is reacted with sodium bicarbonate (NaHCO₃) in a controlled environment. The reaction proceeds as follows:

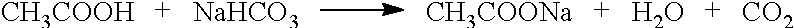

This reaction forms a sodium acetate solution. The sodium acetate solution comprises sodium acetate (CH₃COONa), water (H₂O), and carbon dioxide (CO₂).
(3) Crystallization: The sodium acetate solution obtained from the neutralization reaction is concentrated and then allowed to cool. As the solution cools, sodium diacetate crystals may start to form. The crystals may be separated from the remaining solution through filtration.
(4) Drying: The sodium diacetate crystals are then dried to remove any remaining moisture. This can be done using methods such as air drying or vacuum drying.
(5) Milling and Packaging: Once dried, the sodium diacetate crystals may undergo milling to achieve a uniform particle size. The final product is then packaged in suitable containers for distribution and use in food applications.

It is important to note that the production of sodium diacetate should adhere to food safety and quality standards.

In one embodiment, the ingredients are as follows:
Active Ingredients: Apple Cider Vinegar Blend (Organic Apple; Cider Vinegar, Sodium Diacetate), Zinc Oxide, Vitamin D3;
Inactive Ingredients: Vegan Capsule (Modified Cellulose, Water), Organic Pea Starch, Silica.

The composition is preferably presented as a capsule containing powdered organic apple cider vinegar.

Preferably, the composition does not contain gluten or any of the “Big 8” allergens (wheat, crustacean shellfish, eggs, fish, peanuts, milk, tree nuts, and soybeans) outlined by the FDA. Nor, in a preferred embodiment will it contain any animal products, ingredients, or by-products; it would not have been tested on animals; and the composition would be suitable for vegan and/or vegetarian diet.

One example version of the composition may be as follows:

| | |
|---|---|
| Organic Apple Cider Vinegar Powder 3-8% Acetic Acid | 47.1890 |
| Sodium Diacetate Food Grade Crystal | 47.0630 |
| Organic Vitamin D3 Cholecalciferol 100,000 IU/G | 0.7230 |
| Powder-VegD3 | |
| Zinc Oxide (80% Zn) Powder | 0.8620 |
| Silicon Dioxide Powder | 0.4820 |
| Pea Starch | 3.682 |

### Manufacturing Requirements:

a. Blend ingredients according to formulation;
b. Encapsulate blend into capsules;
c. Personnel inspect capsule weight against specifications and adjust accordingly.
   Filling Requirements, example:
a. Fill by count: 90 capsules (example—can vary);
b. Desiccant strip to be added to bottle;
c. A tamper-evident neckband must be applied over cap and induction sealed liner must be in place;
d. Best By Date and Lot Code must be stamped on each bottle and case.
   Packing Requirements, as example:
Capsule Weight: 800 mg (+/−10%)
Capsules per Bottle: 90
Bottles per Case: 96

### Closing Comments

Throughout this description, the embodiments and examples shown should be considered as examples, rather than limitations on the apparatus and procedures disclosed or claimed. Although many of the examples presented herein involve specific combinations of method acts or system elements, it should be understood that those acts and those elements may be combined in other ways to accomplish the same objectives. With regard to flowcharts, additional and fewer steps may be taken, and the steps as shown may be combined or further refined to achieve the methods described herein. Acts, elements and features discussed only in connection with one embodiment are not intended to be excluded from a similar role in other embodiments.

As used herein, “plurality” means two or more. As used herein, a “set” of items may include one or more of such items. As used herein, whether in the written description or the claims, the terms “comprising”, “including”, “carrying”, “having”, “containing”, “involving”, and the like are to be understood to be open-ended, i.e., to mean including but not limited to. Only the transitional phrases “consisting of” and “consisting essentially of”, respectively, are closed or semi-closed transitional phrases with respect to claims. Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. As used herein, “and/or” means that the listed items are alternatives, but the alternatives also include any combination of the listed items. As used herein, “about” means +/−10% of the stated value.

It is claimed:

1. A composition comprising apple cider vinegar (ACV) in a solid form and a salt of acetic acid, wherein the composition is formulated in a solid dosage form, and wherein the ratio of apple cider vinegar to the salt of acetic acid is in the range of from about 1.5:1 to 0.5:1.

2. The composition as defined in claim 1, wherein the ratio of apple cider vinegar to the salt of acetic acid is in the range from about 1.5:1 to 0.0:1.

3. The composition as defined in claim 1, wherein the ratio of apple cider vinegar to the salt of acetic acid is about 1:1.

4. The composition as defined in claim 1, wherein the salt of the acetic acid is selected from one or more of sodium acetate, calcium acetate, zinc acetate, potassium acetate, sodium di-acetate, potassium di-acetate, acetate, iron acetate, and magnesium acetate.

5. The composition as defined in claim 1, wherein the salt of the acetic acid comprises sodium diacetate.

6. The composition as defined in claim 1, wherein the composition is formulated as one of tablet, gummy, capsule, softgel, or powder sachet dosage form.

7. The composition as defined in claim 1, wherein the ACV is in the form of a dry powder.

8. The composition as defined in claim 1, wherein the salt of acetic acid is produced from vinegar and metal bicarbonate.

9. The composition as defined in claim 8, wherein the vinegar comprises apple cider vinegar.

10. The composition as defined in claim 1, the composition further comprises one or more accessory supplement active ingredients, wherein the accessory supplement active ingredients comprise one or more vitamins, minerals, herbs, amino acids and proteins.

11. The composition as defined in claim 1, the composition comprises:
ACV in an amount of about 313 mg-about 940 mg; and
the salt of the acetic acid in an amount of about 250 mg-about 750 mg.

12. The composition as defined in claim 11, wherein the salt of the acetic acid comprises sodium diacetate.

13. The composition as defined in claim 11, the composition further comprises:
one or more accessory supplement active ingredients in an amount of about 10 mg-about 100 mg.

14. The composition as defined in claim 11, the composition further comprises:
one or more accessory supplement active ingredients in an amount of about 30 mg-about 100 mg.

15. The composition as defined in claim 13, wherein the one or more accessory supplement active ingredients comprise Vitamin D3, Zinc Oxide, Silicon Dioxide powder and Pea Starch powder.

16. The composition as defined in claim 11, the composition further comprises:
Vitamin D3 in an amount about 0.01 mg-about 0.03 mg;
Zinc Oxide in amount about 4.3 mg-about 13 mg;
Silicon Dioxide powder in an amount about 3.2 mg-about 9.6 mg; and
Pea Starch powder in an amount about 24.4 mg-73.3 mg.

17. The composition as defined in claim 1, the composition further comprises an excipient and one more from the group consisting of: Thiamine, Riboflavin, Niacin, Biotin, Pantothenic acid, Pyridoxine, Vitamin B12, Folic Acid, Vitamin C, Vitamin D, Vitamin E, Vitamin K, Calcium, Potassium, Phosphorus, Sodium, Magnesium, Iron, Zinc, Copper, Selenium, Molybdenum, Iodine.

18. The composition as claimed in claim 1, wherein the solid dosage form comprises a capsule containing an efficacious dose of the composition in the range of from about 500 mg to about 2000 mg acetic acid.

19. The composition as claimed in claim 18, wherein an efficacious dose comprises a plurality of capsules in the range of from 1-4.

20. A composition formulated in a dry solid blend comprising approximately 1 to 1 ratio of sodium di-acetate and dry apple cider vinegar powder, an excipient, and one or more discretionary ingredients.
